SEQUENCE LISTING (10 sequences)

SEQ ID NO: 1 - DNA, 381 bp, Bacillus licheniformis ATCC 14580
  CDS (1)..(378)
  mat_peptide (124)..(378)
atgaaaaatcatttgtatgagaaaaaaaagaggaaacctttgactcggacaattaaagcgacgctcgccgtgttgacaatgtccatcgctttggtgggaggcgctacggtgccttcatttgcatgggtgaatccgggttatcactaccagtacccatcggaaggtggtacatggaggtatggattcgtaaacgccgggctccgttcagagtacaaccacccgacaaaggtccacggctcgacagtgcaaaagctcatcgatggaaaagtggataaaacgaatagaagtattgatacggctgcgggccgctactctaatgcctatgtcggagccataaactcacctggtcttaagggtcgttactactatcgcaccaactaa

SEQ ID NO: 2 - protein, 126 aa, Bacillus licheniformis ATCC 14580
MKNHLYEKKKRKPLTRTIKATLAVLTMSIALVGGATVPSFAWVNPGYHYQYPSEGGTWRYGFVNAGLRSEYNHPTKVHGSTVQKLIDGKVDKTNRSIDTAAGRYSNAYVGAINSPGLKGRYYYRTN

SEQ ID NO: 3 - DNA, 1581 bp, Bacillus licheniformis
  CDS (601)..(978)
gaattttccggaagctgaaacacccgtgatatatataaccataaattaaacagcataggcggattgtgcgagttcctccacattcggagtatttctgaatgatagagccacacggtccacgttctcactggctaaccggatcaaatgatcttcaggagtcagcataatacatccagttcaggtagataagatttgaatttggtgacttgcttttgttcttcttctttcattttctgactaatccaaactggaaaaagcaggtcttttaacagattaggaggtttctgacatgcaccattcggtcactaaccgaatgcagtaaaggacactgtggtgcttgccagccattagggtattgaggaggtgatcaaaatgctaggtgacagtatttcgtcgaagtggacaagtcgtgaccaaatgacctcggatcgagggttggtcatggaggaaaaaattgatgtctggtgacaaagaggagtcatgatcatggcaccgccaacgagggaaaaaactcttcccgcatcgacacggtatgtgggcggtgacaaactaacttatagagtaaatttattagtcgaatgaaagaggaggaatgaaataatgaaaaatcatttgtatgagaaaaaaaagaggaaacctttgactcggacaattaaagcgacgctcgccgtgttgacaatgtccatcgctttggtgggaggcgctacggtgccttcatttgcatgggtgaatccgggttatcactaccagtacccatcggaaggtggtacatggaggtatggattcgtaaacgccgggctccgttcagagtacaaccacccgacaaaggtccacggctcgacagtgcaaaagctcatcgatggaaaagtggataaaacgaatagaagtattgatacggctgcgggccgctactctaatgcctatgtcggagccataaactcacctggtcttaagggtcgttactactatcgcaccaactaatcaaagggaaaacggttgctgtcaacggggctagcatggcaagacccagaaaagttctgggagatcccgctttgcataagcgtattatagtggatgacgcgggctttgttgtttacacttcttgcacctgctgacggcaatcatccctatctatgaaatcgagatttcagcaggccgttattttcgagagagttaaatctatattcattgtttttattttggtaaggacataccggattttaggtttggattaccggtcgagttagcttgtcttttcgcccactaccgtgtcgatgcgggagcaatttaccagaagcacttaccgattgatagttttttattccggtgattgcaaagtttcataaactctgagaattcaataggggtaataccccgctttgaggggcgcggcattttatgcgccccgagtatttattcttaaaatttttaaattaatgtatctatataaaaaggagatgctttcggtgtactgccaaagcatctccacaaaagatagtgcatatctgcaggaaaaaacataaaatgcaactaacatttttttggaaagcaataggtttatttaattttgtagttttatct

SEQ ID NO: 4 - protein, 126 aa, Bacillus licheniformis; identical to SEQ ID NO: 2

SEQ ID NO: 5 - protein, 15 aa, Bacillus licheniformis ATCC 14580
  MISC_FEATURE: N-terminal polypeptide sequence
WVNPGYHYQYPSEGG

SEQ ID NO: 6 - DNA, 31 bp, Artificial Sequence
  synthetic primer Pep 481
aattacgcgtgttggtgcgatagtagtaacg

SEQ ID NO: 7 - DNA, 29 bp, Artificial Sequence
  synthetic primer Pep 482
ttaagaattcgaatgaaagaggaggaatg

SEQ ID NO: 8 - protein, 125 aa, Bacillus licheniformis strain 009
  mat_peptide (41)..(125)
MKNLLNKKKRKPLTRTIKATFAVLTVSIGLVGGATVPAFAWVNPDYHYQYPSEGGTWRYGFVNLGLRSEYNHPKKVHGSTVQKLIDGKVEKTNRSLDTAPGRYSNAYVGVVNSPGLKGRYYYRTN

SEQ ID NO: 9 - protein, 126 aa, Bacillus licheniformis strain 470
  mat_peptide (42)..(126)
MKNYLYEKKKRKPLTRTIKATLAVLTMSIALVGGATVPAFAWVNPGYHYQYPSEGGTWRYGFVNAGLRSEYNHPTKVHGSTVQKLIDGKVDKTNRSIDTAAGRYSNAYVGAINSPGLKGRYYYRTN

SEQ ID NO: 10 - protein, 126 aa, Artificial Sequence
  synthetic polypeptide consensus sequence
MKNHLYEKKKRKPLTRTIKATLAVLTMSIALVGGATVPAFAWVNPGYHYQYPSEGGTWRYGFVNAGLRSEYNHPTKVHGSTVQKLIDGKVDKTNRSIDTAAGRYSNAYVGAINSPGLKGRYYYRTN

(19) United States
(12) Patent Application Publication
(10) Pub. No.: US 2010/0098674 A1
(43) Pub. Date: Apr. 22, 2010

(54) USE OF POLYPEPTIDES AS COCCIOSTAT AND/OR HISTOMONASTAT

(76) Inventors: Gilbert Weber, Magden (CH); Jiri Broz, Rheinfelden (CH)

Correspondence Address: NIXON & VANDERHYE, PC, 901 NORTH GLEBE ROAD, 11TH FLOOR, ARLINGTON, VA, 22203, US

(21) Appl. No.: 12/520,593

(22) PCT Filed: Dec. 17, 2007
(86) PCT No.: PCT/EP2007/011059

§ 371 Date: Jul. 13, 2009

(30) Foreign Application Priority Data
Dec. 22, 2006 (EP) 06026709.3
Feb. 5, 2007 (EP) 07002395.7

(51) Int. Cl. A01N 63/00; C07K 14/00; C12N 1/20; A01N 37/18; A01P 15/00

(52) U.S. Cl. 424/93.46; 530/324; 435/252.5; 514/12

(57) ABSTRACT

The present invention relates to the use of isolated polypeptides as a coccidiostat and/or as a histomonastat. An example of a polypeptide of the invention is the so-called L12 protein from Bacillus licheniformis ATCC 14580. The invention furthermore relates to the probiotic use of strains of Bacillus, which produce proteins related to L12.

This invention relates to the use of polypeptides as defined hereinafter in food or animal feed as a coccidiostat and/or histomonastat. An example of a polypeptide of the invention is the so-called L12 protein from Bacillus licheniformis ATCC 14580, which has the amino acid sequence of amino acids +1 to +85 of SEQ ID NO:2 herein (in what follows amino acids 1-85 of SEQ ID NO:2).

Coccidia is a generic name given to single cell protozoan organisms that are intestinal parasites that infect both vertebrates and invertebrates. The organisms cause coccidiosis, and usually settle in the small intestine, such as the colon. Infection with coccidia for farm animals can not only seriously reduce growth, but it can be lifethreatening. Symptoms from coccidial infection include loss of epithelial cells, the denuding of gut mucosa, and diarrhea (often with a concomitant loss of blood). For some farm animals, such as poultry, coccidial infection can be fatal, if not seriously damaging to the animal's health.

Poultry are particularly vulnerable for coccidiosis because of several reasons: (1) The parasitic cycle of 6 to 8 days hits them at a critical stage between weeks 2 and week 4, when maximum growth is usually expressed. Since the parasites virtually destroy the whole intestinal epithelium, the absorption of nutrients is dramatically reduced, which results in marked growth depression. Until slaughter at 5 or 6 weeks, there is not enough time to recover. (2) There are 7 species of Eimeria which can infect poultry, more than in any other animal category, and at least 4 of them are regularly seen in commercial operations. Thus, when one infectious cycle is concluded already another one can be at an early stage so that coccidiosis becomes chronic. (3) In poultry the most pathogenic species (Eimeria tenella, E. necatrix) are observed, which induce severe hemorrhages and in certain cases can cause a mortality of up to 50%. Such an acute case of coccidiosis could easily ruin a poultry farmer. (4) The intensive husbandry of poultry (100,000 chicks or more in one house) on deep litter facilitates access of poultry to the infectious stages of coccidia in the faeces via coprophagy and thus supports a fast spreading of the disease through a whole poultry flock. If the sanitary conditions are not rigorous, the disease will also transfer to other poultry houses on the same farm and stay on site for years.

In order to combat coccidiosis, animal feeds are often supplemented with a coccidiostat. Coccidiostats that have been approved by the EEC for use with poultry (chickens, turkeys, broilers and laying hens) include sulphonimides, amprolium, decoquinate, and ionophores. However, some of these coccidiostats are inorganic compounds that are non-natural and thus have to be made synthetically. This means that they are relatively expensive. There is therefore a need for coccidiostats that are naturally occurring.

Histomonaisis is also caused by a protozoan. In this specific case the protozoa infects the ceca, and later the liver, of turkeys, chickens, and occasionally other galliform birds. In turkeys, most infections are fatal; in other birds, mortality is less common. The protozoan parasite Histomonas meleagridis is transmitted most often in embryonated eggs of the cecal nematode Heterakis gallinarum, and sometimes directly by contact with infected birds. Outbreaks spread quickly through flocks by direct contact. A large percentage of chickens harbor this worm, and histomonads have been located in adult worms of both sexes. Three species of earthworms can harbor H gallinarum larvae containing H meleagridis, which are infective to both chickens and turkeys. H meleagridis survives for long periods within Heterakis eggs, which are resistant and may remain viable in the soil for years. Histomonads are released from Heterakis larvae in the ceca a few days after entry of the nematode and replicate rapidly in cecal tissues. The parasites migrate into the submucosa and muscularis mucosae and cause extensive and severe necrosis. Histomonads reach the liver either by the vascular system or via the peritoneal cavity, and rounded necrotic lesions quickly appear on the liver surface. Histomonads interact with other gut organisms, such as bacteria and coccidia, and depend on these for full virulence.

Traditionally, histomonaisis has been thought of as affecting turkeys, while doing little damage to chickens. However, outbreaks in chickens may cause high morbidity, moderate mortality, and extensive culling. Liver lesions tend to be less severe in chickens, but morbidity can be especially high in young layer or breeder pullets.

In order to combat histomonaisis, animal feeds are often supplemented with a histomonastat. Histomonastats for use with poultry (chickens, turkeys) may include for example 4-nitrophenylarsonic acid a compound that is non-natural and thus has to be made synthetically. Further it is known that the last remaining authorized histomonastat for turkeys, Nifursol, was withdrawn in 2003 by the UK authority. There is therefore also a need for new and additional histomonstats that are for example naturally occurring.

Apart from the usual advantages of using natural compounds, these are likely to be cheaper than synthesising the compound.

The present invention is based on the finding that polypeptides hereinafter defined have activity against coccidiosis and histomoniasis and therefore can be used as coccidiostats and/or histomonastats. Although said polypeptides have already been suggested as additives for animal feed (WO-A-2006/099871), it has not been realised, until now, that these compounds could have been active against coccidia or histomoniasis. Indeed, in WO-A-2006/099871, the polypeptides were instead added to animal feed in order to improve animal feed utilization by improving the feed conversion ratio (FCR), and/or modulating the gut microflora, and there was no mention of any activity against coccidiosis and histomoniasis.

Therefore, this invention relates to the use of a polypeptide selected from the group consisting of:

(a) a polypeptide comprising an amino acid sequence which has a degree of identity to amino acids 1-85 of SEQ ID NO:2 of at least 33%;
(b) a polypeptide which is encoded by a nucleic acid sequence which hybridizes under low stringency conditions with (i) nucleotides 124-378 of SEQ ID NO:1, (ii) a subsequence of (i) of at least 100 nucleotides, or (iii) a complementary strand of (i), or (ii);
(c) a variant of the polypeptide having an amino acid sequence of amino acids 1-85 of SEQ ID NO:2 comprising a substitution, deletion, extension, and/or insertion of one or more amino acids;
(d) an allelic variant of (a) or (b); and
(e) a fragment of (a), (b), (c), or (d).
as a coccidiostat and/or histomonastat.

By using a polypeptide as defined above as a coccidiostat for example, one can employ a naturally occurring compound which is more likely to be acceptable to the human or animal being treated for coccidiosis. Also, industry and consumer groups are often in favour of using naturally occurring compounds rather than synthetic ones. The polypeptides are organic, and may therefore be cheaper to provide than synthetic inorganic compounds.

In particular embodiments the polypeptide has, consists essentially of, or consists of an amino acid sequence which has a degree of identity to amino acids 1-85 of SEQ ID NO:2 of at least 33%, such as, e.g., the polypeptide of amino acids 1-85 of SEQ ID NO:2. Other to specific examples are the polypeptides of amino acids 1-85 of any one of SEQ ID NOs:8, 9, and 10 (identified as L12-likes on the basis of the PCR-test of Example 1 herein).

A polypeptide of the present invention may be a bacterial or a fungal polypeptide. In a second particular embodiment, the polypeptide is a Gram positive bacterial polypeptide such as a Bacillus polypeptide or a variant thereof, for example a Bacillus licheniformis polypeptide e.g. derived from Bacillus licheniformis ATCC 14580, which is the type strain of Bacillus licheniformis and available on request from the American Type Culture Collection, ATCC. Preferred strains of Bacillus licheniformis are positive in the test of Example 1 herein, such as the following strains of Bacillus licheniformis: ATCC 14580 (=NCIB 9375), NCIMB 6346 (=DSM 8785), NCTC 1024, NCTC 1025, NCTC 2120, NCTC 7589, NCTC 9932, ATCC 21424, NCIMB 10689, and ATCC 53757.

A second aspect of the present invention relates to the use of polypeptides as defined hereinabove in the preparation of a coccidiostatic or histomonastatic composition.

A further aspect of the invention relates to an animal feed composition, such as suitable for a monogastric or non ruminant animal, comprising a polypeptide as defined hereinabove which is present as a coccidiostat and/or as a histomonastat. The polypeptide according to the invention is preferably present in an amount at which it has coccidiostatic activity or is active against coccidia.

The term animal includes all animals. Examples of other animals than poultry are ruminant animals including for example sheep, goat, and cattle, e.g. cow such as beef cattle and dairy cows. In a particular embodiment, the animal is a non-ruminant animal. Non-ruminant animals include pet animals, e.g. cats or dogs and mono-gastric animals, e.g. in addition to poultry pig or swine (including, but not limited to, piglets, growing pigs, and sows); fish (including but not limited to salmon, trout, tilapia, catfish and carp); and crustaceans (including but not limited to shrimp and prawn).

A further aspect of the invention relates to a premix or additive composition, such as to be added to one or more edible feed substance (s) or ingredient (s), for example to prepare or for supplementation to an existing feed to form a feed composition.

According to the invention the polypeptide can be used as isolated pure polypeptide or in a mixture of polypeptides.

As defined herein, an “isolated” or “pure” polypeptide is a polypeptide which is essentially free of other polypeptides, e.g., at least 80% pure, preferably at least 85%, 86%, 87%, 88%, 89%, or at least 90% pure, more preferably at least 91%, 92%, 93%, 94%, 95%, or at least 96% pure, as determined by SDS-PAGE (e.g., by coomassie-staining and subsequent scanning by methods known in the art). Purity may also be determined by HPLC, preferably RP-HPLC (e.g., using a Waters μ-Bondapak C18 column, Mobil phase A: 0.1% TFA, Mobil phase B: Acetonitrile+0.1% TFA, detecting at 280 nm). The SDS-PAGE purity, as well as the HPLC purity, refers to the amount of the polypeptide of the invention, relative to the amount of total protein. In alternative embodiments, the polypeptide may be at least 20%, 40%, 60%, or at least 70% pure.

The amount of total protein can be determined by any method known in the art, e.g. the Kjeldahl method (A.O.A.C., 1984, Official Methods of Analysis 14th ed., Association of Official Analytical Chemists, Washington D.C.), and the amount of the polypeptide of the invention can be determined by SDS-PAGE and subsequent scanning, also by methods known in the art.

Polypeptides encoded by nucleic acid sequences of the present invention also include fused polypeptides or cleavable fusion polypeptides in which another polypeptide is fused at the N-terminus or the C-terminus of the polypeptide or fragment thereof. A fused polypeptide is produced by fusing a nucleic acid sequence (or a portion thereof) encoding another polypeptide to a nucleic acid sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

In a specific embodiment, the polypeptide is a low-allergenic variant, designed to invoke a reduced immunological response when exposed to animals, including man. The term immunological response is to be understood as any reaction by the immune system of an animal exposed to the polypeptide. One type of immunological response is an allergic response leading to increased levels of IgE in the exposed animal. Low-allergenic variants may be prepared using techniques known in the art. For example the polypeptide may be conjugated with polymer moieties shielding portions or epitopes of the polypeptide involved in an immunological response. Conjugation with polymers may involve in vitro chemical coupling of polymer to the polypeptide, e.g. as described in WO 96/17929, WO98/30682, WO98/35026, and/or WO99/00489. Conjugation may in addition or alternatively thereto involve in vivo coupling of polymers to the polypeptide. Such conjugation may be achieved by genetic engineering of the nucleotide sequence encoding the polypeptide. Another way of providing low-allergenic variants is genetic engineering of the nucleotide sequence encoding the polypeptide so as to cause the polypeptides to self-oligomerize, effecting that polypeptide monomers may shield the epitopes of other polypeptide monomers and thereby lowering the antigenicity of the oligomers. Such products and their preparation is described e.g. in WO 96/16177. Epitopes involved in an immunological response may be identified by various methods such as the phage display method described in WO 00/26230 and WO 01/83559, or the random approach described in EP 561907. Once an epitope has been identified, its amino acid sequence may be altered to produce altered immunological properties of the polypeptide by known gene manipulation techniques such as site directed mutagenesis (see e.g. WO 00/26230, WO 00/26354 and/or WO 00/22103) and/or conjugation of a polymer may be done in sufficient proximity to the epitope for the polymer to shield the epitope.

In just a further aspect, the invention relates to the use in animal feed of a strain of Bacillus, the DNA of which, when harvested and used as a DNA template in a PCR reaction with SEQ ID NOs:6 and 7 as primers, leads to the generation of a PCR fragment of a size of approximately 0.4 kb. This test serves to identify strains with an L12-like gene, see Example 1 herein. These strains of Bacillus may also be used in the preparation of a composition for use in animal feed as coccidiostat.

In a first particular embodiment, the Bacillus strain is a probiotic microorganism. The term “probiotic” generally refers to a non-pathogenic bacterium fed to animals, including birds, as a way to prevent colonization by pathogenic microorganisms, e.g. protazoa. Probiotics may also be defined as live, or livable, micro-organisms which beneficially affect the intestinal balance of healthy and normally functioning humans and animals.

In a second particular embodiment, the Bacillus strain is used in the form of spores. Spores may be exospores or, preferably, endospores. An endospore is any spore that is produced within an organism (usually a bacterium). Endospores can survive through periods of environmental stress, and are therefore capable of surviving passage of the harsh (acid) environment of the upper gastro intestinal tract, while only exerting its effect once it reaches the intestines, where normal vegetative cells will be formed.

In further particular embodiments, the strain of Bacillus is a strain of Bacillus licheniformis, preferably selected from the following strains of Bacillus licheniformis: ATCC 14580 (=NCIB 9375), NCIMB 6346 (=DSM 8785), NCTC 1024, NCTC 1025, NCTC 2120, NCTC 7589, NCTC 9932, ATCC 21424, NCIMB 10689, and ATCC 53757. A preferred subgroup includes Bacillus licheniformis ATCC 14580 (=NCIB 9375), and Bacillus licheniformis NCIMB 6346 (=DSM 8785).

The test of Example 1 is a PCR reaction, in this example conducted with DNA isolated from various strains of Bacillus licheniformis. In a particular embodiment of this test, the DNA used as template for the PCR reaction is chromosomal DNA which can be isolated by methods known in the art. The result of the Example 1 test is positive when a PCR fragment of the right size is obtained. In Example 1, the right size is indicated as 0.4 kb. In a particular embodiment, the right size is between 0.35 kb and 0.44 kb (=350 bp-440 bp). In alternative embodiments, the right size is 330-430 bp, 340-420 bp, 350-410 bp, 360-400 bp, 370-390 bp, or 385-395 bp. The size of the coding sequence (CDS) of SEQ ID NO:1 is approximately 380 bp (viz. 378 bp).

Isolated nucleic acid sequences encoding the polypeptides as defined hereinabove, nucleic acid constructs, vectors and host cells comprising the nucleic acid sequences for expressing and production of isolated polypeptides as hereinabove defined as well as probiotic strains as exemplified hereinabove are described in WO-A-2006/099871. The content of this publication, in particular variants of polypeptides and strains according to the inventions and their production, is hereby incorporated by reference.

The polypeptide compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art.

Particular examples of compositions of the invention are the following:
An animal feed additive comprising (a) a polypeptide of the invention; and (b) at least one fat-soluble vitamin, (c) at least one water-soluble vitamin, (d) at least one trace mineral, and/or (e) at least one macro mineral;
an animal feed composition having a crude protein content of 50 to 800 g/kg and comprising a polypeptide of the invention;
an animal feed additive comprising (a) a strain of Bacillus as defined in the section headed “Bacterial strains; Probiotic Bacillus strains”; and (b) at least one fat-soluble vitamin, (c) at least one water-soluble vitamin, (d) at least one trace mineral, and/or (e) at least one macro mineral; and
an animal feed composition having a crude protein content of 50 to 800 g/kg and comprising a strain of Bacillus as defined in the section headed “Bacterial strains; Probiotic Bacillus strains”.

The so-called premixes are examples of animal feed additives of the invention. A premix designates a preferably uniform mixture of one or more micro-ingredients with diluent and/or carrier. Premixes are used to facilitate uniform dispersion of micro-ingredients in a larger mix.

The term feed or feed composition means any compound, preparation, mixture, or composition suitable for, or intended for intake by an animal.

In the use according to the invention the polypeptide and/or the Bacillus strain can be fed to the animal before, after, or simultaneously with the diet. The latter is preferred.

In a particular embodiment, the polypeptide, in the form in which it is added to the feed, or when being included in a feed additive, is well-defined. The term well-defined means that the polypeptide preparation is at least 50% pure. In other particular embodiments the well-defined polypeptide preparation is at least 60, 70, 80, 85, 88, 90, 92, 94, or at least 95% pure.

A well-defined polypeptide preparation is advantageous. For instance, it is much easier to dose correctly to the feed a polypeptide that is essentially free from interfering or contaminating other polypeptides. The term dose correctly refers in particular to the objective of obtaining consistent and constant results, and the capability of optimising dosage based upon the desired effect.

For the use in animal feed, however, the polypeptide need not be that pure; it may e.g. include other polypeptides such as animal feed enzymes, in which case it could be termed a polypeptide preparation.

The polypeptide preparation can be (a) added directly to the feed (or used directly in a treatment process of proteins), or (b) it can be used in the production of one or more intermediate compositions such as feed additives or premixes that is subsequently added to the feed (or used in a treatment process). The degree of purity described above refers to the purity of the original polypeptide preparation, whether used according to (a) or (b) above.

Polypeptide preparations with purities of this order of magnitude are in particular obtainable using recombinant methods of production, whereas they are not so easily obtained and also subject to a much higher batch-to-batch variation when the polypeptide is produced by traditional fermentation methods.

The polypeptide can be added to the feed in any form, be it as a relatively pure polypeptide, or in admixture with other components intended for addition to animal feed, i.e. in the form of animal feed additives, such as the so-called pre-mixes for animal feed.

A part from the polypeptide and/or the Bacillus strain of the invention, the animal feed additives of the invention contain at least one fat-soluble vitamin, and/or at least one water soluble vitamin, and/or at least one trace mineral, and/or at least one macro mineral.

Further, optional, feed-additive ingredients are colouring agents, e.g. carotenoids such as beta-carotene, astaxanthin, and lutein; aroma compounds; stabilisers; antimicrobial peptides; other coccidiostats; reactive oxygen generating species; and/or at least one enzyme selected from amongst phytase (EC 3.1.3.8 or 3.1.3.26); xylanase (EC 3.2.1.8); galactanase (EC 3.2.1.89); alpha-galactosidase (EC 3.2.1.22); protease (EC 3.4., phospholipase A1 (EC 3.1.1.32); phospholipase A2 (EC 3.1.1.4); lysophospholipase (EC 3.1.1.5); phospholipase C (EC 3.1.4.3); phospholipase D (EC 3.1.4.4); amylase such as, for example, alpha-amylase (EC 3.2.1.1); and/or beta-glucanase (EC 3.2.1.4 or EC 3.2.1.6).

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of other coccidiostats which may also be used are ionophores such as lasalocid, monensin, salinomycin, maduramycin, semduramycin and chemical agents as amprolium, nicarbazin, diclazuril.

As disclosed in WO-A-2003/009700 polyunsaturated fatty acids have been discovered to have coccidiostatic activity as well. Therefore, in a particular preferred embodiment of the invention further feed-additive ingredients are polyunsaturated fatty acids (PUFAs).

The (animal feed) composition may comprise from 0.001, 0.01 g or 1 g, up to 0.01 or 100 g of PUFA per kg feed, preferably from 0.0001 to 100 g/kg. Amounts may be as low as from 0.0001 up to 0.1 g of PUFA per kg of feed, for example from 0.0025 (or 0.05 or 0.08) to 0.001 (or 0.01 g) of PUFA per kg of feed. Typically, the composition will comprise from 0.002 to 0.01 g of PUFA per kg of feed, preferably from 0.0004 g to 0.08 g of PUFA per kg of feed.

The above amounts refer to the weight of the PUFA, and so if the PUFA is added in the form of an oil (for example having from 30 to 40% of the PUFA), then the amount of oil present (or added) can be calculated accordingly, for example by multiplying the amount of the PUFA by 100/X where X is the percentage of the PUFA in the oil. Hence, for example with a 30 (or 35) to 40 (or 45 or 50%) PUFA content, the amount of oil that can be added may vary proportionally, such as from 0.00033 or 0.00025 g up to 330 or 250 g of oil per kg of feed.

The PUFA can either be a single PUFA or two or more different PUFAs. Each PUFA can be of the n-3 or n-6 family. Preferably it is a C18, C20 or C22 PUFA. It may have at least 18 carbon atoms and 3 double bonds. The PUFA can be provided in the form of a free fatty acid, a salt, as a fatty acid ester (e.g. methyl or ethyl ester), as a phospholipids and/or in the form of a mono-, di- or triglyceride.

Suitable (n-3 and n-6) PUFAs include:
docosahexaenoic acid (DHA, 22: 6Ω3), suitably from algae or fungi, such as the (dinoflagellate) Crypthecodinium or the (fungus) Thraustochytrium;
y-linolenic acid (GLA, 18: Ω6;
a-linolenic acid (ALA, 18: Ω3);
conjugated linoleic acid (octadecadienoic acid, CLA);
dihomo-y-linolenic acid (DGLA, 20: Ω6);
arachidonic acid (ARA, 20: Ω6); and
eicosapentaenoic acid (EPA, 20: 5 Ω3).

Preferred PUFAs include arachidonic acid (ARA), docosohexaenoic acid (DHA), eicosapentaenoic acid (EPA) and/or y-linoleic acid (GLA). In particular, ARA is preferred.

The PUFA may be from a natural (e.g. vegetable or marine) source or may be derived from a single cell or microbial source. In particular, the PUFA may be produced by a bacteria, alga, fungus or yeast. Fungi are preferred, preferably of the order Mucorales, for example Mortierella, Phycomyces, Blakeslea, Aspergillus, Thraustochytrium, Pythium or Entomophthora. The preferred source of ARA is from Mortierella alpina, Blakeslea trispora, Aspergillus terreus or Pythium insidiosum. Algae can be dinoflagellate and/or include Porphyridium, Nitszchia, or Crypthecodinium (e.g. Crypthecodinium cohnii). Yeasts include those of the genus Pichia or Saccharomyces, such as Pichia ciferii. Bacteria can be of the genus Propionibacterium.

The PUFA may be present in or be added to the composition as an (e.g. edible) oil. The oil may be a liquid (at room temperature). The oil may be a microbial (e.g. single cell), marine (e.g. tuna) oil or a vegetable oil. A suitable oil that includes ARA is available from DSM N. V., Alexander Fleminglaan 1 or Wateringseweg 1, P.O. Box 1,2600 MA Delft, The Netherlands, under the trade mark VEVODAR. Another commercially available (ARA) oil is ARASCO from Martek Corporation, 6480 Dobbin Road, Columbia, Md. 21045, United States of America. Other PUFAs are available, for example DHA as a DHA oil (DHASCO from Martek Corporation or DHA from Pronova, Norway, under the trademark PAX).

A number of documents describe the production of crude PUFA oils. Microbial oils containing ARA are disclosed in WO-A-92/13086 (Martek), EPA in WO-A-91/14427 (Martek) and DHA in WO-A-91/11918 (Martek). Various methods for extracting PUFA oils from microbial sources can be found in WO-A-97/36996 and WO-A-97/37032 (both Gist-brocades). Preparation of ARA, DHA and EPA-containing oils is also described in WO-A-92/12711 (Martek).

It is preferred that most of the PUFA is in the form of triglycerides. Thus, preferably at least 50%, such as at least 60%, or optimally at least 70%, of the PUFA is in triglyceride form. However, the amount of triglycerides may be higher, such as at least 85%, preferably at least 90%, optimally at least 95% or 98% of the oil.

Preferably the additive or premix comprises from 10 to 1,000, such as from 25 or 50 to 750, preferably from 75 or 100 to 250 or 500, times as much of the PUFA (or other components, such as enzymes) as the feed. This is because the premix can be “diluted” by a factor of 10 to 1,000 (so that the premix constitutes from 10% to 0.1% of the final feed) when making the animal feed. Any of these figures can thus be used to multiply the (maximum and/or minimum) values of the amounts of the PUFA as set out in the next section to obtain concentration (s) of the PUFA in the premix. As a broad range however, the concentration may be from 1 to 100 g/kg. The premix may be in the form of granules or pellets.

Examples of reactive oxygen generating species are chemicals such as perborate, persulphate, or percarbonate; and enzymes such as an oxidase, an oxygenase or a syntethase.

Usally fat- and water-soluble vitamins, as well as trace minerals form part of a so-called premix intended for addition to the feed, whereas macro minerals are usually separately added to the feed. Either of these composition types, when enriched with a polypeptide or a Bacillus strain of the invention, is an animal feed additive of the invention.

The following are non-exclusive lists of examples of these components:
Examples of fat-soluble vitamins are vitamin A, vitamin D3, vitamin E, and vitamin K, e.g. vitamin K3.
Examples of water-soluble vitamins are vitamin B12, biotin and choline, vitamin B1, vitamin B2, vitamin B6, niacin, folic acid and panthothenate, e.g. Ca-D-panthothenate.
Examples of trace minerals are manganese, zinc, iron, copper, iodine, selenium, and cobalt.
Examples of macro minerals are calcium, phosphorus and sodium.

The nutritional requirements of these components (exemplified with poultry and piglets/pigs) are listed in Table A of WO 01/58275. Nutritional requirement means that these components should be provided in the diet in the concentrations indicated.

Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore such fish diets usually have a crude fat content of 200-310 g/kg.

WO 01/58275 corresponds to U.S. Ser. No. 09/779,334 which is hereby incorporated by reference.

An animal feed composition according to the invention has a crude protein content of 50-800 g/kg, and furthermore comprises at least one polypeptide and/or at least one Bacillus strain as described and/or claimed herein.

In a particular embodiment, the animal feed composition of the invention contains at least one vegetable protein or protein source. It may also contain animal protein, such as Meat and Bone Meal, and/or Fish Meal, typically in an amount of 0-25%. The term vegetable proteins as used herein refers to any compound, composition, preparation or mixture that includes at least one protein derived from or originating from a vegetable, including modified proteins and protein-derivatives. In particular embodiments, the protein content of the vegetable proteins is at least 10, 20, 30, 40, 50, or 60% (w/w).

Vegetable proteins may be derived from vegetable protein sources, such as legumes and cereals, for example materials from plants of the families Fabaceae (Leguminosae), Cruciferaceae, Chenopodiaceae, and Poaceae, such as soy bean meal, lupin meal and rapeseed meal. In a particular embodiment, the vegetable protein source is material from one or more plants of the family Fabaceae, e.g. soybean, lupine, pea, or bean. In another particular embodiment, the vegetable protein source is material from one or more plants of the family Chenopodiaceae, e.g. beet, sugar beet, spinach or quinoa.

Animal diets can e.g. be manufactured as mash feed (non pelleted) or pelleted feed. Typically, the milled feed-stuffs are mixed and sufficient amounts of essential vitamins and minerals are added according to the specifications for the species in question. The polypeptide(s) and/or the Bacillus strain can be added as solid or liquid formulations. For example, a solid polypeptide formulation is typically added before or during the mixing step; and a liquid polypeptide preparation is typically added after the pelleting step. The polypeptide may also be incorporated in a feed additive or premix.

The final polypeptide concentration in the diet is within the range of 0.01-200 mg protein per kg diet, for example in the range of 0.1-20 mg protein per kg animal diet.

The polypeptide and/or the Bacillus strain should of course be applied in an effective amount, i.e. in an amount adequate for improving feed conversion.

It is at present contemplated that the polypeptide is administered in one or more of the following amounts (dosage ranges): 0.01-200; 0.01-100; 0.5-100; 1-50; 5-100; 10-100; 0.05-50; 1-10; or 0.10-10, all these ranges being in mg polypeptide protein per kg feed (ppm). In a particular embodiment, the dosage range is 1-9, 1-8, 2-7, 2-6, or 2-5 ppm. Examples of particularly preferred dosage range are; 0.5-15.0, 1.0-12.5, 1.5-10.0, and 2.5-7.5 ppm.

It is at present contemplated that the Bacillus strain is administered in one or more of the following amounts (dosage ranges): 10 E2-14, 10 E4-12, 10 E6-10, 10 E7-9, preferably 10 E8 CFU/g of feed (the designation E meaning exponent, viz., e.g., 10 E2-14 means 102-1014).

For determining mg polypeptide protein per kg feed, the polypeptide is purified from the feed composition, and the dosage in mg polypeptide protein per kg feed is calculated, for example as described in Example 10 of WO-A-2006/099871. The same principles apply for determining mg polypeptide protein in feed additives.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### EXAMPLE 1

### Bacillus Strains with L12-Like Genes, as Identified by PCR

Genes similar to the gene encoding the L12 protein (SEQ ID NO:1) were identified in a number of other Bacillus licheniformis strains by PCR. DNA for use as a template for the PCR reaction was isolated from eleven different Bacillus licheniformis strains grown overnight at 37° C. on TY agar plates. One inoculation tube with cells from each strain were suspended in 0.1 ml H₂O and boiled for 10 min, centrifuged, and 5 microliter supernatant from each was used as DNA template in PCR reactions as described below.

The PCR reactions were run in “Pure Taq™ Ready-To_Go™ PCR Beads” from Amersham Biosciences: 5 microliter DNA template+2×1 microliter of primer Pep481 (SEQ ID NO:6) and Pep482 (SEQ ID NO:7)+18 microliter H2O.

PCR program: 1) 95° C. 3 min; 2) 95° C. 10 sec; 3) 65° C. 30 sec −1° C. pr. cycle; 4) 72° C. 1 min; 5) Go To 2) 9 times; 6) 95° C. 10 sec; 7) 55° C. 30 sec; 8) 72° C. 1 min; 9) Go To 6) 19 times; 10) 72° C. 5 min; 11) 4° C. forever, which means that following step 10) the temperature is lowered to 4° C.

| | |
|---|---|
| Primers: | |
| Pep481 | |
| AATTACGCGTGTTGGTGCGATAGTAGTAACG-3′ | (SEQ ID NO: 6) |
| Pep482 | |
| TTAAGAATTCGAATGAAAGAGGAGGAATG-3′ | (SEQ ID NO: 7) |

The resulting 0.4 kb PCR fragment from five positive strains (positive meaning giving DNA band of the right size) were purified and used in a DNA sequencing experiment, using once again as sequence primers the Pep481 (SEQ ID NO:6) and Pep482 (SEQ ID NO:7) primers.

Three of the five positive strains gave the same DNA sequence: Bacillus licheniformis ATCC 14580, Bacillus licheniformis NCIMB 6346 (=DSM 8785) and Bacillus licheniformis strain 712, resulting in the amino acid sequence of SEQ ID NO:2. In Bacillus licheniformis strain 470 DNA changes resulted in two amino acid changes (SEQ ID NO:9), however none in the mature peptide. In Bacillus licheniformis strain 009 DNA changes resulted in fifteen amino acid changes (SEQ ID NO:8), eight of which in the mature peptide. Furthermore, a consensus sequence (SEQ ID NO:10) was derived from SEQ ID NOs:2, 8 and 9.

Note that, in this experiment, the nucleotides encoding the seven C-terminal amino acids of SEQ ID NO:2 are included in the Pep481 primer (SEQ ID NO:6), and the seven C-terminal amino acid residues of SEQ ID NOs:8-9 may therefore not be correct. However the correctness of SEQ ID NOs:8-9 was later confirmed.

A strain of Bacillus licheniformis which was isolated from the in-feed probiotic product designated BioPlus™2B (offered by Chr. Hansen A/S, 10-12 Boege Allé, DK-2970 Hoersholm, Denmark) was included amongst the eleven strains tested but was negative.

In addition, 44 other strains of Bacillus licheniformis were tested as described above. A positive PCR-response was found in 27 of these strains. Examples of additional publicly available strains of Bacillus licheniformis found to be L12-positive have the following deposit numbers: NCTC 1024, NCTC 1025, NCTC 2120, NCTC 7589, NCTC 9932, ATCC 21424, NCIMB 10689, ATCC 53757. NCTC is the National Collection of Type Cultures. ATCC is the American Type Culture Collection. NCIMB is the National Collection of Industrial, Marine and Food Bacteria.

### EXAMPLE 2

### Animal Feed and Additive

### Animal Feed Additive

An animal feed additive is prepared by adding 25 g of a coated T-granulate comprising the purified L12 protein in an amount of 20 g/kg (prepared as described in Example 3 of EP 569468 B1, however with a coating of approx. 7% hydrogenated palm oil and approx. 13% CaCO3) to the following premix (per kilo of premix):

| | | |
|---|---|---|
| 1100000 | IE | Vitamin A |
| 300000 | IE | Vitamin D3 |
| 4000 | IE | Vitamin E |
| 250 | mg | Vitamin B1 |
| 800 | mg | Vitamin B2 |
| 1200 | mg | Ca-D-Panthothenate |
| 500 | mg | Vitamin B6 |
| 2.5 | mg | Vitamin B12 |
| 5000 | mg | Niacin |
| 10000 | mg | Vitamin C |
| 300 | mg | Vitamin K3 |
| 15 | mg | Biotin |
| 150 | mg | Folic acid |
| 50004 | mg | Cholin chloride |
| 6000 | mg | Fe |
| 3000 | mg | Cu |
| 5400 | mg | Zn |
| 8000 | mg | Mn |
| 124 | mg | I |
| 60 | mg | Co |
| 29.7 | mg | Se |
| 9000 | mg | Lasalocid Sodium (Avatec) |

| | |
|---|---|
| 17.3% | Ca |
| 0.8% | Mg |
| 11.7% | Na |

### Animal Feed

A broiler grower diet having the following composition (%, w/w) is prepared by mixing the ingredients. Wheat, rye and SBM 48 are available from Moulin Moderne Hirsinque, Hirsingue, France. After mixing, the feed is pelleted at a desired temperature, e.g. about 70° C. (3×25 mm).

| | |
|---|---|
| Wheat | 46.00 |
| Rye | 15.00 |
| Soy Bean Meal (SBM 48) | 30.73 |
| Soybean oil | 4.90 |
| DL-Methionine | 0.04 |
| DCP (Di-Calcium Phosphate) | 1.65 |
| Limestone | 0.43 |
| Salt | 0.15 |
| TiO2 | 0.10 |
| Animal feed additive (above) | 1.00 |

The resulting animal feed comprises 5.0 mg purified L12 protein per kg (5 ppm).

Additional animal feed and feed additive compositions are prepared in the same manner, however substituting 25 g coated L12 CT granulate per kg of the premix with 1013 Colony Forming Units (CFU), preferably in the form of endospores, of Bacillus licheniformis ATCC 14580, which results in an animal feed with approximately 108 CFU per g of the feed composition.

1. The use of an isolated polypeptide selected from the group consisting of:
(a) a polypeptide comprising an amino acid sequence which has a degree of identity to amino acids 1-85 of SEQ ID NO:2 of at least 33%;
(b) a polypeptide which is encoded by a nucleic acid sequence which hybridizes under low stringency conditions with
(i) nucleotides 124-378 of SEQ ID NO: 1,
(ii) a subsequence of (i) of at least 100 nucleotides, or
(iii) a complementary strand of (i), or (ii);
(c) a variant of the polypeptide having an amino acid sequence of amino acids 1-85 of SEQ ID NO:2 comprising a substitution, deletion, extension, and/or insertion of one or more amino acids;
(d) an allelic variant of (a) or (b); and
(e) a fragment of (a), (b), (c), or (d);
as or for the preparation of a coccidiostat and/or histomonastat.

2. Use in animal feed of a strain of Bacillus, the DNA of which, when harvested and used as a DNA template in a PCR reaction with SEQ ID NOs:6 and 7 as primers, leads to the generation of a PCR fragment of a size of approximately 0.4 kb, as a coccidiostat and/or histomonastat.

3. Use of a coccidiostat and/or histomonastat according to claim 2 in animal feed.

4. Use according to claim 1, wherein the isolated polypeptide is combined with at least one polyunsaturated fatty acid (PUFA), which has coccidiostatic activity and/or is active against coccidia.

5. A method of treatment or prophylaxis of coccidiosis and/or histomonaisis comprising administering an animal feed or feed additive containing a polypeptide according to claim 1.

6. An animal feed composition, or an additive or premix composition therefore, comprising a polypeptide according to claim 1 as a coccidiostat and/or histomonastat.

7. A composition according to claim 6 which further comprises at least one polyunsaturated fatty acid (PUFA).

8. An additive or premix composition for an animal feed composition, comprising a polypeptide according to claim 1 as a coccidiostat.

9. Use of a coccidiostat and/or histomonastat according to claim 1 in animal feed.

10. A method of treatment or prophylaxis of coccidiosis and/or histomonaisis comprising administering an animal feed or feed additive containing a strain according to claim 2.

11. An animal feed composition, or an additive or premix composition therefore, comprising a strain of Bacillus according to claim 2 as a coccidiostat and/or histomonastat.

12. A composition according to claim 11 which further comprises at least one polyunsaturated fatty acid (PUFA).

13. An additive or premix composition for an animal feed composition, comprising a strain of Bacillus according to claim 2 as a coccidiostat.
